SEQUENCE LISTING (2 sequences)

SEQ ID NO: 1 - DNA, 584 bp, Homo sapiens
  CDS (11)..(409)
  ref <301>: AUTHORS: Kanetsky,P.A., Swoyer,J., Panossian,S.,
  ref <302>: TITLE: A polymorphism in the agouti signaling protein gene is associated with human pigmentation.
  ref <303>: JOURNAL: Am. J. Hum. Genet.
  ref <304>: VOLUME: 70
  ref <305>: ISSUE: 3
  ref <306>: PAGES: 770-775
  ref <307>: DATE: 2002-02-06
  ref <308>: DATABASE ACCESSION NUMBER: NM_001672
  ref <309>: DATABASE ENTRY DATE: 2003-04-03
  ref <308>: DATABASE ACCESSION NUMBER: NM_001672
  ref <309>: DATABASE ENTRY DATE: 2003-12-20
  ref <313>: RELEVANT RESIDUES: (11)..(409)
gcctcctgggatggatgtcacccgcttactcctggccaccctgctggtcttcctctgcttcttcactgccaacagccacctgccacctgaggagaagctccgagatgacaggagcctgagaagcaactcctctgtgaacctactggatgtcccttctgtctctattgtggcgctgaacaagaaatccaaaccgatcggcagaaaagcagcagaaaagaaaagatcttctaagaaggaggcttcgatgaagaaagtggtgcggccccggacccccctatctgcgccctgcgtggccacccgcaacagctgcaagccgccggcacccgcctgctgcgacccgtgcgcctcctgccagtgccgcttcttccgcagcgcctgctcctgccgcgtgctcagcctcaactgctgagcgcccccactcccggccgcgagcaggcagggcttcggggacgcggggcgcttctcgggcgggtgatccctaacagggcggcttcccagggctgcaggcgggcggaggttccaggagatgggacttcagggagacctggcttgggctaaaatcgaaatacaatatatataggctg

SEQ ID NO: 2 - protein, 132 aa, Homo sapiens [sequence omitted]

(19) United States
(12) Patent Application Publication
(10) Pub. No.: US 2005/0032735 A1
(43) Pub. Date: Feb. 10, 2005

(54) Method for regulating the skin and hair color in a post-natal mammal

(75) Inventors: Bin-Ru She, Cyonglin Township (TW); Lin-Cheng Yang, Kaohsiung City (TW); Chih-Hsun Yang, Taoyuan County (TW); Ping-Ching Wu, Kaohsiung City (TW)

Correspondence Address: BACON & THOMAS, PLLC, 625 SLATERS LANE, FOURTH FLOOR, ALEXANDRIA, VA, 22314, US

(73) Assignee: Industrial Technology Research Institute, Hsinchu (TW)

(21) Appl. No.: 10/860,481

(22) Filed: Jun. 4, 2004

(30) Foreign Application Priority Data
Aug. 8, 2003 (TW) 092121806

(51) Int. Cl. A61K 48/00; A61K 7/06

(52) U.S. Cl. 514/44; 424/70.13

(57) ABSTRACT

A method for regulating the skin and hair color in a post-natal mammalian by preparing a recombinant vector in which an agouti-encoding DNA segment is positioned under the control of a promoter and introducing the recombinant vector into skin cells of the mammalian. The recombinant vector carrying the genes expresses related proteins after they are introduced to most mammalian cells.

### BACKGROUND OF THE INVENTION

1. Field of the Invention

The present invention relates to a method for regulating the skin and hair color in a post-natal mammalian and, more particularly, to a method for regulating the skin and hair color in a post-natal mammalian via gene therapy.

2. Description of Related Art

The treatment of human hair or skin by bleaching, dying, physical or chemical abrasion, high-energy light therapy to improve hair or skin color has generally been unsatisfactory. Those treatments usually have their limitations and side-effects. In particular, some studies have indicated that coloring hair with various dyes may lead to increased tumor susceptibility. Thus, this invention provides an alternative method for regulating the skin and hair color in a post-natal mammalian.

2.1 Melanin

Melanin, the pigment produced in melanocytes of the skin cells, is largely responsible for the coloring of skin and hair. It is synthesized in melanosomes from the amino acid tyrosine into dihydroxyphenylalanine (DOPA) and dopaquinone.

The tyrosinase is the key enzyme for melanin biosynthesis and is required in these early steps. After the tyrosinase steps, the pathways to produce different forms of melanins diverge and involve many other enzymes.

Generally, there are two major forms of melanin in all pigmented animals—eumelanin and pheomelanin. Eumelanin is brown to black in color while pheomelanin is yellow to red in color. The relative amount of the two forms of melanin is the major determinant of hair and skin color. (Thody A. J. and Graham A., Pigment Cell Res. 11:265-274, 1998)

The regulation of the production of eumelanin versus pheomelanin involves the interaction of the melanocortin 1 receptor (MC1R) on the surface of the melanocyte with either the alpha-melanocyte stimulating hormone (α-MSH) or the agouti signaling protein (ASP). Binding of MSH to MC1R results in the formation of eumelanin while the binding of the ASP to MC1R leads to the production of pheomelanin. (Suzuki, I., Ollman, M., et.al., J. Invest. Dermatology, 108:838-842, 1997)

MC1R is a G-protein-coupled receptor, i.e. it uses proteins that bind guanosine triphosphate (GTP) and guanosine diphosphate (GDP) as an intermediary messenger. Following the binding of α-MSH to GTP-G_{sα} subunit then activates adenylate cyclase, leading to increased production of cyclic adenosine monophosphate (cAMP) within the melanocyte. An increase in the intracellular concentration of cAMP leads to an increase in tyrosinase activity and eumelanin production.

ASP is an antagonist of α-MSH at the MC1R. In the skin, ASP is produced by follicular melanocytes, and it acts as a paracrine factor to control whether eumelanin or pheomelanin is produced. ASP can abrogate the stimulatory effects of α-MSH on cAMP formation and tyrosinase activity; and further, it can inhibit alpha-MSH-induced eumelanin production, resulting in the subterminal band of pheomelanin often visible in mammalian skin. (Kanetsky PA. et al, Am. J. Hum. Genet. 70:770-775, 2002) In addition, ASP has been shown to down-regulate genes necessary for eumelanogenesis (Abdel-Malek Z. et al., Proc. Natl. Acad. Sci. USA 92:1789-1793, 1995)

2.2 Agouti Gene

The agouti gene is present in most mammals, e.g. dogs, foxes, mice and humans. The agouti gene product regulates production of eumelanin and pheomelanin.

The mouse and human agouti genes have been cloned and sequenced. The mouse agouti gene encodes a distinctive 131 -amino acid protein with a consensus signal peptide. Sequence analysis revealed that the coding region of the human agouti gene is 85% identical to the mouse gene and has the potential to encode a protein of 132 amino acids with a consensus signal peptide. (Heajoon Y. Kwon et al, Proc. Natl. Acad. USA 91:9760-9764,1994)

In the past, most research on the agouti gene was focused on the relationship between this gene product and metabolism which increases the susceptibility to obesity, diabetes and hypertension. Some of that research have shown that when this gene was introduced into a mouse embryonic stem cell, the mouse derived from the embryonic stem cell displayed type II diabetes symptoms. (Klebig et al, Proc. Natl. Acad. Sci. USA 92:4728-4732,1995); Kucera et al. (Dev. Biol. 173:162-173,1996). demonstrated that ectopic-expression of this gene in the mouse skin, using transgenic method, did not result in a syndrome of obesity and insulin resistance. However, the method used in the study can be applied only at the early embryo stage. Therefore, these studies implied that some serious problems exist in the method for introducing the agouti gene into the post-natal animals. Nevertheless, the present invention overcomes deficiencies in the prior art and describes the unexpected results obtained by the inventors that the method for introducing the agouti gene into mammalian can successfully be applied to post-natal animals without the undesirable side-effects such as diabetes, hyperinsulinemia and obesity.

### SUMMARY OF THE INVENTION

The object of the present invention is to provide a method for regulating the skin and hair color in a mammalian that can be applied to post-natal animals without encountering adverse effects such as diabetes, hyperinsulinemia and obesity.

To achieve the object, the method comprises introducing into the skin cells of the mammalian a DNA fragment encoding a protein involved in the regulation of melanin synthesis, such that the DNA fragment is expressed in a sufficient number of skin cells of the mammalian to regulate the skin and hair color in a mammalian.

The present invention further provides methods of gene therapy, wherein the DNA fragment encodes a protein which modulates production of pheomelanin. Such a protein will increase production of pheomelanin, thereby lightening the skin and hair color.

Other objects, advantages, and novel features of the invention will be apparent from the following detailed description, taken in conjunction with the accompanying drawings.

### BRIEF DESCRIPTION OF THE DRAWINGS

FIG. 1 is a diagram illustrating a vector (pCMV plasmid vector) and the inserted fragment (hASP) in the example of the invention.

FIG. 2 shows the changes in rat's skin or hair color after pCMVhASP was delivered into skin (indicated by an arrow) with gene-gun method.

FIG. 3 shows the changes in rat's skin or hair color from day 0 to day 28 after pCMVhASP was delivered into skin with the gene-gun method.

FIG. 4 shows the changes in rat's skin or hair color measured by Mexameter from day 0 to day 28 after delivery of pCMVhASP to skin with the gene-gun method.

FIG. 5 shows the Western blotting of the proteins prepared from the skin several days after delivery of pCMVhASP to skin cells with the gene-gun method; wherein antibodies used were specific for ASP, α-tubulin, MC1R and tyrosinase respectively.

FIG. 6 shows the immunohistochemistry of rat's skin tissue several days after delivery of pCMVhASP to skin with the gene-gun method, wherein antibodies used were specific for ASP or tyrosinase.

FIG. 7 shows the effect of agouti-induced weight gain monitored regularly between 0 and 4 weeks of gene gun injection.

FIGS. 8a and 8b show changes in rat's skin or hair color (indicated by an arrow) after pCMVhASP was delivered into the skin via direct injection combined with PEI.

FIG. 9 shows the Western blotting of the proteins prepared from the skin tissue several days after pCMVhASP or pCMVEGFP was delivered into skin via direct injection combined with PEI.

### DETAILED DESCRIPTION OF THE DRAWINGS

The present invention provides a method of gene therapy for regulating the skin and hair color in a mammalian through the delivery into, and expression in, a DNA fragment of the skin cells of a mammal, the fragment encoding a protein involved in the modulation of eumelanin and pheomelanin.

The present invention specifically provides a method of gene therapy wherein the DNA fragment is the nucleic acid sequence in which which the agouti gene product is expressed. The product can control whether eumelanin or pheomelanin is produced. As used herein, an “agouti gene” means a nucleic acid sequence encoding an agouti protein or peptide. Preferred agouti genes include mammalian agouti genes, and in particular those from humans and mice. A preferred nucleic acid sequence encoding an agouti gene is the nucleotide sequence of SEQ ID NO: 1.

Regarding the agouti-encoding nucleotide sequence, the present invention encompasses the nucleic acid sequences that may be synthetic DNA sequences or isolated natural DNA sequences, or any functionally equivalent nucleic acid sequences, analogs and portions thereof and encode one or more proteins having agouti activity as describe herein. The DNA sequences may also be complementary DNA (cDNA) or genomic DNA. As will be understood by those skilled in the art, the DNA sequence can easily be synthesized by chemical techniques, for example, phosphotriester method (Matteucci, et al., J. Am. Chem. Soc. 103:3185-3191,1981) or using automated synthesis methods.

The present invention further provides a method of gene therapy wherein the DNA fragment is delivered by means of a recombinant vector. The recombinant vector of the present invention may also contain a nucleotide sequence encoding suitable regulatory elements, so as to effect expression of the vector construct in skin cells. As used herein, “expression” refers to the ability of the vector to transcribe the inserted DNA fragment into MRNA so that synthesis of the protein encoded by the inserted nucleic acid can occur. Those skilled in the art will appreciate the following: (1) that a variety of promoters and enhancers are suitable for use in the constructs of the invention; and (2) constructs will contain the necessary start, termination, and control sequences for proper transcription and processing of the DNA fragment encoding a protein involved in the regulation of melanin synthesis, on introduction of recombinant vector construct into the skin cells of the mammalian.

The vectors provided by the present invention, for the expression in skin cells of the DNA fragment encoding a protein in the regulation of melanin synthesis, may comprise the following vectors known to one skilled in the art: phage, cosmid, baculovirus, retroviral, plasmid and yeast artificial chromosome (YAC) vectors. Other vectors would be apparent to one skilled in the art. In a preferred embodiment, the vector of the present invention used is the pCMV plasmid vector.

In a recombinant expression vector, the coding portion of the DNA segment is positioned under the control of a promoter. The promoters may include agouti promoters themselves, or promoters normally associated with other genes, and in particular other transcription factor genes, or promoters isolated from any bacterial, viral, eukaryotic, or mammalian cell. Naturally, it will be important to employ a promoter that effectively directs the expression of the agouti-encoding DNA segment in the cell type, organism, or even animal, chosen for expression. The use of promoter and cell type combinations for protein expression are generally known to those of skill in the art of molecular biology, for example, see Sambrook et al. (1989). The promoters employed may be constitutive, or inducible, and can be used under the appropriate conditions to direct high level expression of the introduced DNA segment, such as is advantageous in the large-scale production of recombinant proteins or peptides. This particular embodiment of the present invention provides for regulation of expression of the DNA fragment encoding the protein, through the use of constitutive promoter. The promoter for use in the recombinant vectors of the present invention is the CMV early gene promoter.

Transdermal delivery of therapeutic agents, such as peptides, proteins, and other biomolecules, has been used successfully for several decades. In the present invention, the introduction into the skin cell of a recombinant vector containing the DNA fragment may be effected by suitable methods known to one skilled in the art, such as DEAE Dextran, cationic liposome or polyethylenimine (PEI) mediated delivery, viral-vector mediated delivery, DNA-coat microprojectile bombardment (gene gun), microprojection patch, iontophoresis, skin abrasion and naked DNA transfer by, for example, direct injection. It will be appreciated by those skilled in the art that any of these methods of DNA transfer may be combined (Lieb et al, J. Pharmaceutical Sci. 86:1022-1029, 1997 ; Brus et al, J. Controlled Release 84:171-181, 2002 ; Lin et al, Pharm Res. 18:1789-1793,2001; Ausubel, F. M.et al., Current Protocols in Molecular Biology, New York, 1992 ; and Sambrook, et al., Molecular Cloning, A Laboratory Manual, Cold Spring Harbor Laboratory Press, New York, 1989).

In a preferred embodiment of the present invention, the DNA fragment is delivered into the skin cells of the mammalian by DNA-coated microprojectile bombardment. The DNA-coated microprojectile bombardment techniques have been applied for DNA vaccination in cancer, or pain gene therapy. Helium gas forced DNA coated microscopic gold particles (0.5 to 3 um) can be targeted to the epidermal skin layer and thus are suitable for skin gene therapy. The injection area (1 to 50 cm²) and depth can be adjusted, depending on the particle size, helium gas pressure and the characteristics of target tissue. The particles can be injected into cells or between cells. The gold particles are non-toxic and seldom induce immune responses. Gene-gun mediated delivery has been verified to effectively deliver DNA to various organs, tissues, and cells (Huang, L. et al, 1999, Nonviral vectors for gene therapy, Academic Press).

In another embodiment of the present invention, the DNA fragment is delivered into the skin cells of the mammalian by direct injection. The DNA fragment may be combined with PEI. Cationic polymer PEI is condensed with anionic polymer DNA to form a PEI/DNA complex, which enters the cell through endocytosis. The introduced gene can express after the endosome breaks and releases the DNA. It was suggested that the amines in PEI buffer protons in the endosome, result in the influx of Cl⁻, osmotic swelling and the consequent endosomelysis (Godbey W.T. et al, 1999, Proc. Natl. Aca. Sci. USA. 96:5177-5181). Advantages of PEI mediated delivery include high DNA stability, high nuclear target efficiency, and high gene expression efficiency.

The practice of the present invention employs, unless otherwise indicated, conventional molecular biological techniques, which are within the skill of the art. See e.g., “Molecular Cloning: A Laboratory Manual”, second edition (Sambrook et al., 1989); “Oligonucleotide Synthesis” (M. J. Gait, ed., 1984); “Gene Transfer Vectors for Mammalian Cells” (J. M. Miller & M. P. Calos, eds., 1987); “Current Protocols in Molecular Biology” (F. M. Ausubel et al., eds., 1987); “PCR: The Polymerase Chain Reaction”, (Mullis et al., eds., 1994).

The present invention is described in the following examples. That section is set forth to aid in the understanding of the invention, and should not be construed to limit in any way the invention as defined in the claims which follow thereafter.

### EXAMPLES

1. Cloning of the human agouti signaling protein (hASP) cDNA (Genbank Accession No. NM_{—}001672) into the pCMV plasmid vector

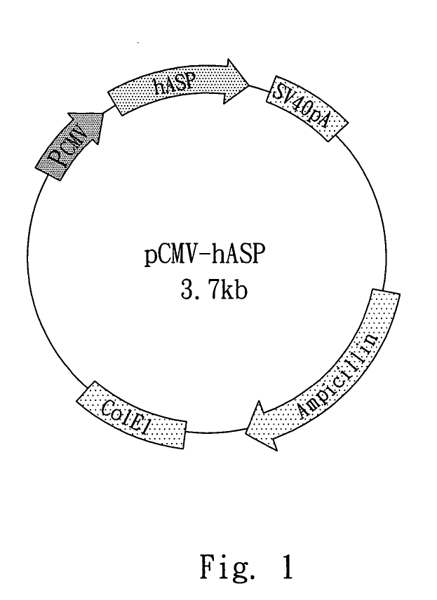

The nucleotide sequence of the human agouti signaling protein cDNA is available at Genbank Accession No. NM_{—}001672 and is disclosed in SEQ ID NO: 1 herein. The pCMV plasmid was purchased from Strategen. The HASP cDNA (approximately 584 nucleotides long) was inserted into the EcoRi cloning site of pCMV plasmid vector to construct pCMV-hASP (approximately 3.7 kb long) (FIG. 1), where expression is driven off the CMV early gene promoter. The pCMV-hASP construct was subsequently amplified by being transformed into Escherichia coli via standard methods and purified with Maxi plasmid purification kit (Qiagen).
2. Gene transfer of the hASP gene into skin cells of the Long-Evans (LE) rats through DNA-coat microprojectile bombardment (gene gun)

The pCMV-hASP construct was prepared as described above. 105 μg of pCMV-hASPs were coated on 28 mg of gold particles (1.5-2 μm in diameter) and injected into the skin cells of LE male rats (approximately 300-350 g weight) via Helio gene gun system (Bio-Rad), each pulse injecting 0.5 mg of gold particles and 1.875 μg of DNA. Control rats were injected with pCMVEGFP DNA. The site of injection was the dark spot on the dorsal skin of the LE rat.

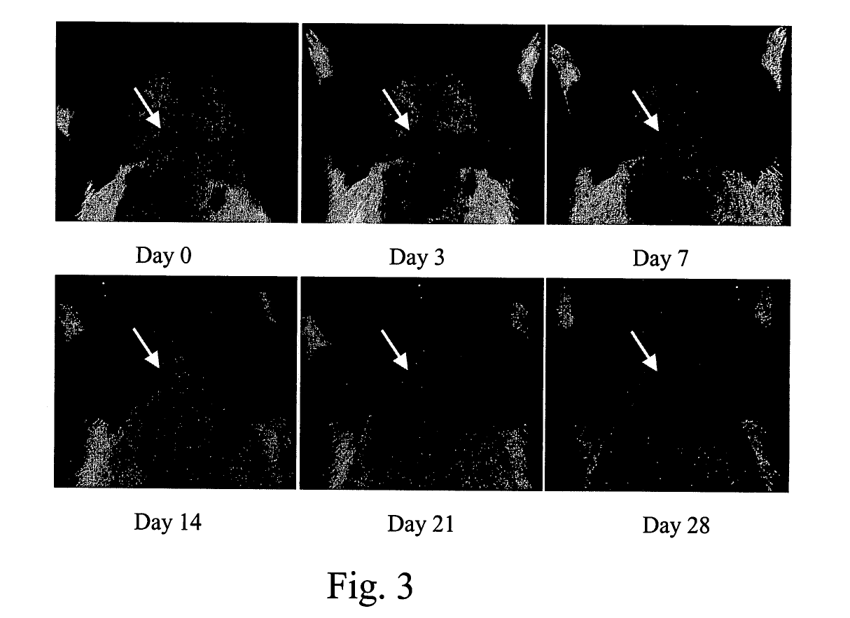

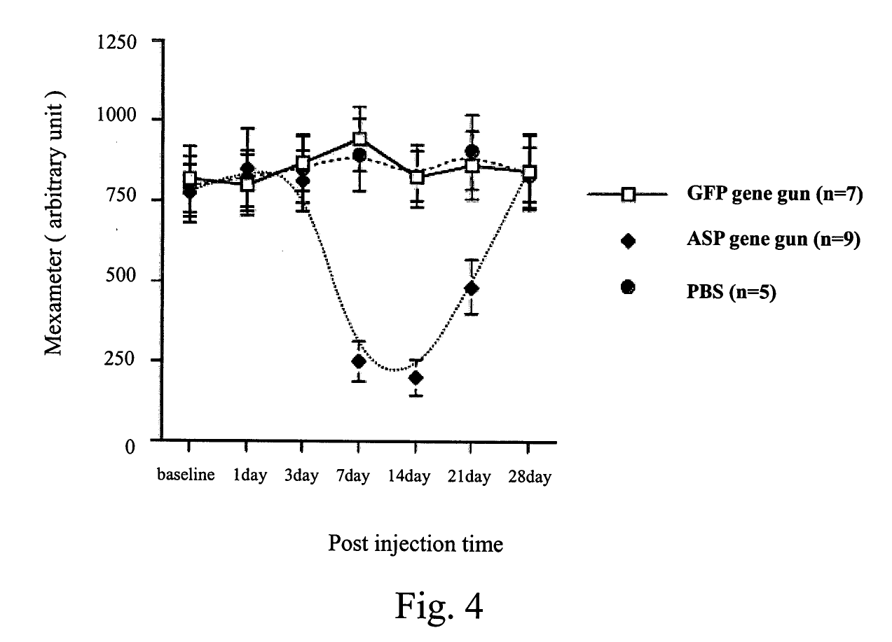

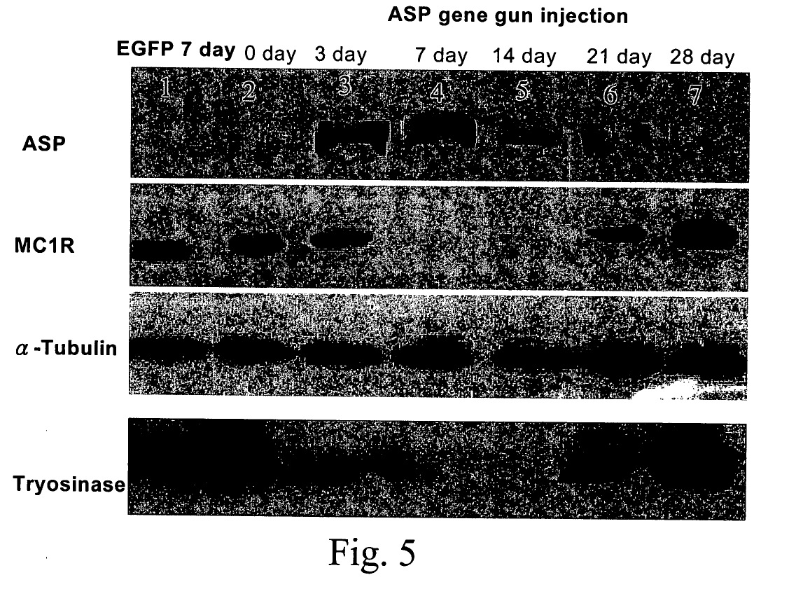

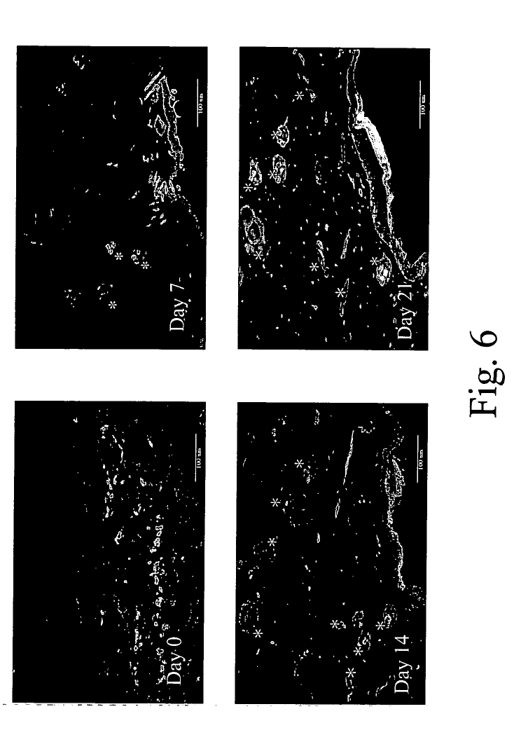

After a period of time, the skin and hair color in rats were altered as shown in FIG. 3 and FIG. 4. Besides, a Mexameter MX18 (Courage+Khazaka Electronic GMBH, Koln, Germany) was used to objectively quantify changes in the color of skin (FIG. 4). The relative amounts of ASP, MC1R, α-tubulin and tyrosinases were determined by Western blotting method(see FIG.5). Agouti signaling protein production in gene-gun-treated skin tissue was further confirmed by immunohistochemical analysis (FIG. 6), wherein sections of skin injected with pCMVhASP were positive for ASP immunoreactivity.

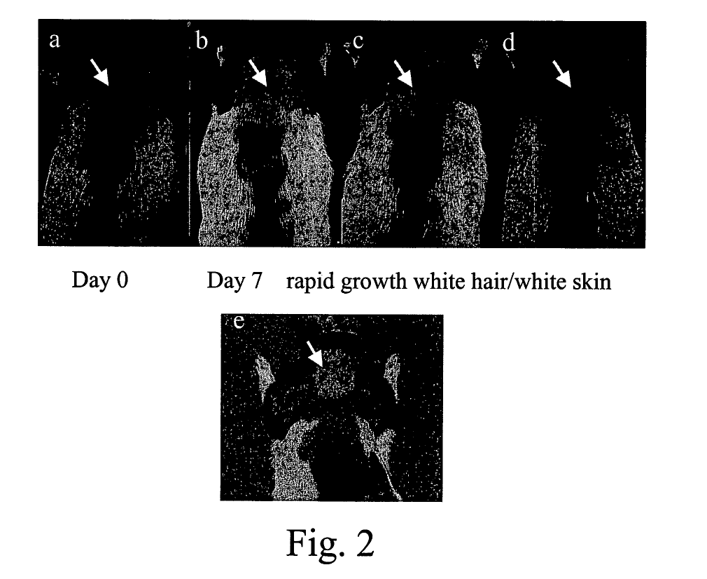

With reference to FIG. 2, the hair color of an LE rat before injection of pCMVhASP is shown in panel A; the hair color of the LE rat 7 days after injection of pCMVhASP is shown in panel B; the injection site shown in panel B was shaved or partially shaved to reveal the skin color,shown in panels C and D; and the hair color of the LE rat 7 days after injection of pCMVEGFP is shown in panel E. The results demonstrate that the skin or hair color lightened after injection of pCMVhASP (panels A to D) which was not observed after the injection of pCMVEGFP (panel E).

Please also refer to FIG. 3, which shows that the color change in rats started on day 3, maximized in week 2 and recovered after 4 weeks.

Referring to FIG. 4, a Mexameter MX18 (Courage+Khazaka Electronic GMBH, Koln, Germany) was used to objectively quantify changes in the skin color of rats. The dorsal skin color of LE rats was measured at baseline and day 1, 3, 7, 14, 21 and 28 after gene gun injection. ASP cDNA gene gun injection decreased the level of pigmentation of skin (P<0.05) while the control groups, GFP cDNA and PBS injections, did not induce any obvious change in the skin color of rats.

With reference to FIG. 5, which shows the Western blotting analysis using antibodies specific to ASP, MC1R, α-tubulin and tyrosinas. Skin biopsies injected with pCMVhASP or pCMVEGFP were taken at the time points indicated. Levels of agouti signal protein in the skin of ASP cDNA treated animals increased from day 0 on, reached maximum on day 7, then decreased to basal level on day 28. (Lane 2-7). The ASP level did not increase in the GFP cDNA treated group (Lane 1) (P<0.05). Importantly, both the levels of MC1-R and tyrosinase decreased following the increase of ASP level, which might have caused the change in skin pigmentation.

In FIG. 6 (a) light spots showed the fluorescence image of tyrosinase expression. Samples bombarded with ASP cDNA on days 0, 7, 14, and 21 showed increased ASP expression (showed with star marks) and decreased tyrosinase expression. In contrast, sections taken from the GFP cDNA treated animals and control sections from which the primary antibody was omitted, displayed no immunoreactivity (data not shown). These results indicated a good correlation in the relative activities measured by histochemical staining and mexameter.

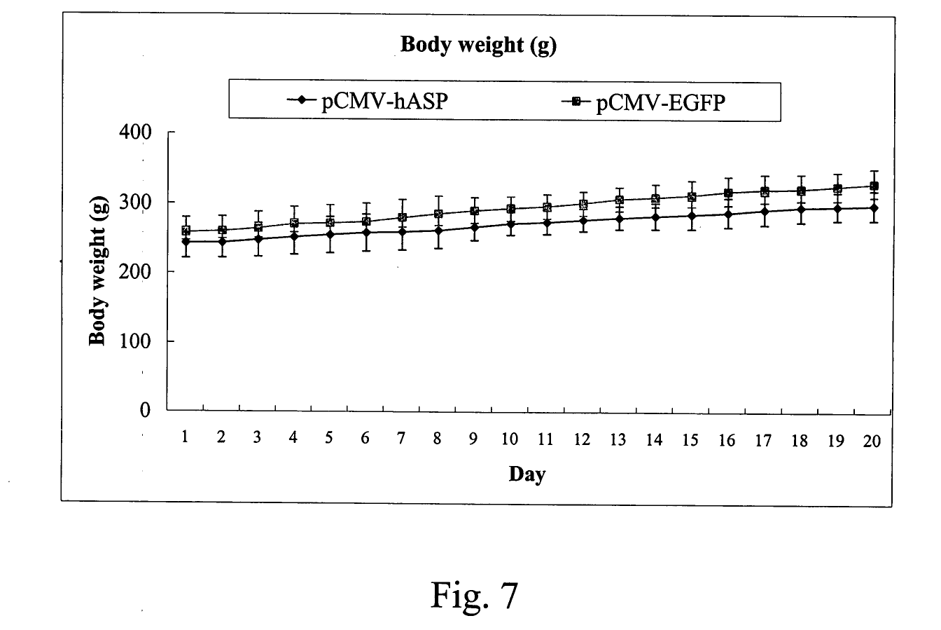

To study the effect of agouti-induced weight gain, the weight gain of the animals was monitored regularly between 0 and 4 weeks of gene gun injection (see FIG.7). Weight growth curves also were determined for pCMV GFP, pCMV ASP, and PBS treated rats. ASP gene gun injection was not found to increase the weight gain in the rats during the 4-week period studied. All weight curves were not significantly different as analyzed by ANOVA with multiple measurements (P>0.05). No differences in blood glucose concentrations were found among thesegroups (data not shown).
3. Gene transfer of the hASP gene into skin cells of the Long-Evans (LE) rats through direct injection combined with PEI.

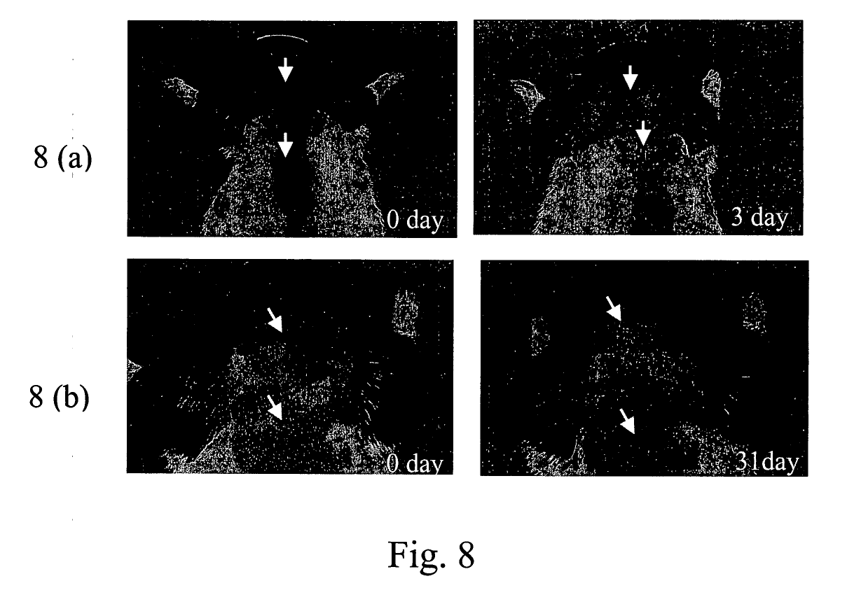

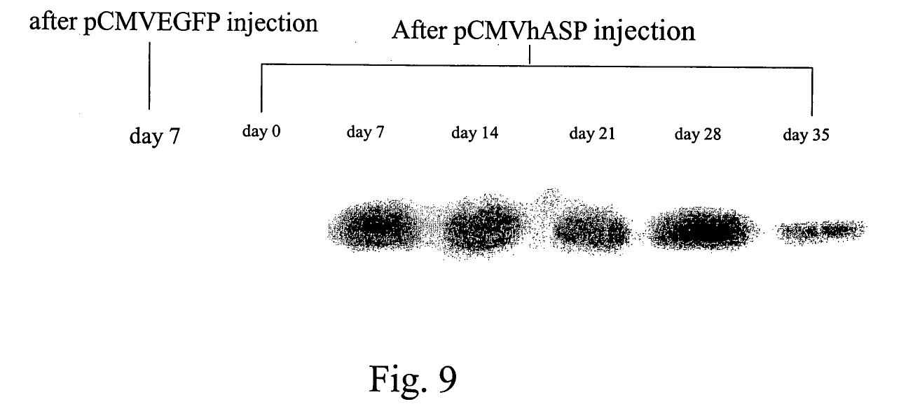

The pCMV-hASP construct was prepared as described above. 45 mg of branched PEI (Sigma, average MW is 25,000) was dissolved in 10 ml of D5W (pH 6.5) (PEI conc. was about 100 mM or 0.45 %). 1 μg of pCMVhASP DNA mixed with 0.3 μl of PEI solution (N/P of PEI/DNA is 10) stood at room temperature for 20 min and injected into the skin cells of LE male rats (approximately 300 g -350 g weight) using insulin syringe. Control rats were injected with pCMVEGFP DNA. The sites of injection were the dark spots on the dorsal skin of LE rats. After a period of time, the hair or skin color in the ASP cDNA treated rats were altered as shown in FIG. 8a and FIG. 8b. Besides, the relative amounts of ASP were determined by Western blotting method (see FIG. 9).

With reference to FIG. 8a and 8b, the discolor of skin started on day 3 after injection of pCMVhASP, and remained for up to 31 days.

With reference to FIG. 9, which shows the Western blotting analysis using an antibody specific to ASP, skin biopsies injected with pCMVhASP or pCMVEGFP were taken at the time points indicated. Levels of agouti signal protein in the skin of ASP cDNA treated animals increased from day 0 on, remained for up to 31 days. The ASP level did not increase in the GFP cDNA treated group.

In the present invention, a method for delivering human ASP cDNA via gene therapy method into the skin of Long-Evans (LE) rats to alter hair and skin color is disclosed. The results show that local cutaneous transfer of ASP plasmid using gene therapy method can alter mice skin color without changing feeding behavior or body weight. In addition, the method of the present invention is suitable for application on confined tissue in post-natal animals without encountering adverse effects such as diabetes, hyperglycemia and obesity.

Although the present invention has been explained in relation to its preferred embodiment, it is to be understood that many other possible modifications and variations can be made without departing from the spirit and scope of the invention as hereinafter claimed.

1. A method for regulating the skin and hair color in a post-natal mammalian, comprising the steps of:
(a) preparing a recombinant vector in which an agouti-encoding DNA segment is positioned under the control of a promoter;
(b) introducing said recombinant vector into skin cells of the mammalian;
wherein said recombinant vector carrying said genes expresses related proteins after they are introduced to most mammalian cells.

2. The method as claimed in claim 1, further comprising a step (b1) of amplifying and purifying said recombinant vector prepared from step (a) and then proceeding to step (b).

3. The method as claimed in claim 1, wherein said agouti-encoding DNA segment is the nucleic acid sequence of mammalian agouti gene.

4. The method as claimed in claim 1, wherein said agouti-encoding DNA segment is the nucleic acid sequence of humans or mouse agouti gene.

5. The method as claimed in claim 1, wherein said agouti-encoding DNA segment is the nucleic acid sequence of a human agouti gene.

6. The method as claimed in claim 1, wherein said agouti-encoding DNA segment is the nucleic acid sequence of SEQ ID NO: 1.

7. The method as claimed in claim 1, wherein said vector is phage, cosmid, baculovirus, retroviral, plasmid or yeast artificial chromosome (YAC) vectors.

8. The method as claimed in claim 1, wherein said vector is pCMV plasmid vector.

9. The method as claimed in claim 1, wherein said promoter is the constitutive promoter.

10. The method as claimed in claim 1, wherein said promoter is the CMV early gene promoter.

11. The method as claimed in claim 1, wherein said recombinant vector is introducing into the skin cells of the mammalian by at least one method selected from the group consisting of DEAE Dextran, cationic liposome or polyethylenimine (PEI) mediated delivery, viral-vector mediated delivery, DNA-coat microprojectile bombardment, microprojection patch, iontophoresis, skin abrasion and direct injection.

12. The method as claimed in claim 1, wherein said recombinant vector is introduced into the skin cells of the mammalian by microprojectile bombardment.

13. The method as claimed in claim 1, wherein said recombinant vector is introduced into the skin cells of the mammalian by direct injection.

14. The method as claimed in claim 1, wherein said recombinant vector is introducing into the skin cells of the mammalian by direct injection combined with PEI.
